(19) United States
(12) Patent Application Publication
(10) Pub. No.: US 2010/0040565 A1
(43) Pub. Date: Feb. 18, 2010

(54) ACTIVE DELIVERY SYSTEMS FORMULATIONS

(75) Inventors: Andrew M. HOMOLA, Naples, FL (US); R. Gary Pitts, Nevada City, CA (US); Ronald K. Dunton, Guemes Island, WA (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: Castle Beach, LLC, Carson City, NV (US)

(21) Appl. No.: 12/540,201

(22) Filed: Aug. 12, 2009

Related U.S. Application Data
(60) Provisional application No. 61/104,129, filed on Oct. 9, 2008.
(60) Provisional application No. 61/088,155, filed on Aug. 12, 2008.

(51) Int. Cl. A61K 8/18; A61K 33/40; A61K 33/06; A01N 53/00; A61Q 17/04; A61Q 11/00

(52) U.S. Cl. 424/49; 424/59; 424/616; 424/682; 514/531

(57) ABSTRACT

The present invention relates to active delivery system formulations, and methods of making and using the same. Said formulations, when applied to a substrate surface, form a protective coating on the surface and permit constituent active agents to act on the surface and in the surrounding medium.

### BACKGROUND OF THE INVENTION

1. Field of the Invention

The present invention relates to active delivery system formulations that, when applied to a substrate surface, form a protective coating on the surface and permit constituent active agents to act on the surface and in the surrounding medium.

2. Description of the Background

With respect to the delivery of active agents to the surface of teeth, the oral care industry and health research communities have looked for many years for a way to interdict the attachment, propagation, growth or colonization of bacteria on teeth since adhered bacteria are the start of a pernicious chain of events leading to formation of home care-resistant plaque, calculus, and ultimately, tooth-loss. As people in developed countries live longer, dental care plays a larger role in overall health, and developing countries are becoming more aware of the importance of oral hygiene.

Dental plaque results when cariogenic bacteria (e.g., Streptococcus mutans) collect in colonies and form deposits on tooth surfaces. The presence of the bacteria and deposits is extremely detrimental to the health of the tooth and the surrounding gingival tissue for, if left unchecked, they may result in infected gingival tissue, the formation of dental caries and possibly periodontal disease. In extreme cases their presence may even result in the loss of teeth. Many attempts have been made to control or prevent both the occurrence of dental caries and the formation of dental plaque. For example, fluoride solutions or gels have been used. However, none of the conventional approaches provide a completely satisfactory delivery system. Furthermore, similar to the unsatisfactory conventional dental delivery systems, known delivery systems for the delivery of a myriad of active agents to a multitude of substrates are also unsatisfactory.

For example, with respect to the delivery of active agents to skin, e.g., insect repellant and sunscreen formulations, the powders, sprays, solutions, lotions and creams for many forms of dermatological conditions lack effectiveness. The reason for this can vary, from poor delivery of the active agent to the source or cause of the condition, to loss of the active agent through abrasion from normal activity of the patient, to absorption of active agents applied to the skin by the patient's socks or clothes, etc. For these various reasons, active agents are prone to come off easily once applied to the affected area, and consequently much of the active agent is wasted, either through over application in an attempt to anticipate the problem, or in the active agent quickly being dispersed away from the site. Thus the compositions containing the active agents typically require continuous application, and thus often fail due to this poor delivery. Furthermore, in applications in which dosing is important, reliably dispensing a properly measured dose of the treatment is often difficult by its very nature, and is made even more difficult when the compositions delivering the active agents may be removed before the treatment is complete.

In addition, bandages and adhesive patches have been used to deliver active agents to body surfaces in a manner that reduces premature removal of the active agent, allows more reliable dosing, and reduces mess. However, such treatment devices are often bulky and therefore may be uncomfortable for the user. Also, removal of the bandage or adhesive patch from the body surface after treatment is often uncomfortable or even painful.

Hence, with respect to dermal applications, there is a need for a clean and inexpensive vehicle/carrier of topically applied medications/compositions that increases the convenience and effectiveness of the treatment (i.e., delivery of the active agents), decreases the necessary time for the treatment, and provides a single device capable of delivering a broad range of treatments/active agents to a variety of body surfaces. It is preferably associated with less waste and lower cost and ultimately leads to improved treatment of dermal conditions and increased patient satisfaction.

Accordingly, in view of the conventional delivery systems there remains a definite need for improved methods, compositions, and delivery systems which are effective for, e.g., the prevention, amelioration and/or treatment of infection, contamination and/or bacterial adhesion to substrates. There also remains a need for (i) preventing, ameliorating and/or treating the diseases and conditions which result from such infestation or infection and/or (ii) application of cosmetic or other treatments to substrates.

### SUMMARY OF THE INVENTION

Employing the compositions of U.S. Pat. No. 5,980,868, the disclosure of which is incorporated herein by reference in its entirety, the present inventors have surprisingly discovered that it is possible to obtain improved effects from active agents in said compositions. Such improved effects can be obtained by providing the active agents on carriers before the active agents are incorporated into said compositions including cationic surface active transfer agents and hydrophobic barrier-forming materials.

More specifically, since the '868 patent, it has been discovered by the present inventors that the efficacy of the active agents in the compositions of the '868 patent may be less than desired for at least the two following reasons: (1) the active agent becomes surrounded and isolated by the other components of the composition and is therefore significantly prevented from being delivered to the desired substrate, and (2) as the concentration of the active agent is increased, the active agent inhibits the attachment/adhesion of the composition to the substrate.

In view of this problem the present inventors have discovered that providing the active agents on and into carriers before the active agents are incorporated into compositions including cationic surface active transfer agents and hydrophobic barrier-forming materials permits the use of higher volumes or dosages of the active agents and, thus, more enhanced active agent performance than would be otherwise possible. The compositions and methods of the present invention allow for the protection of biological and non-biological substrates (hereinafter sometimes referred to as simply “substrates”) and the provision of additional effects from a wide range of active agents. For example, such effects can include the prevention, amelioration and/or treatment of a wide variety of diseases and other conditions; modification of sensation; and/or the inhibition or reduction of pain.

In the context of this disclosure, the term prevent or prevention refers to being capable of reducing the likelihood and/or delaying the onset of a particular disease/condition. In addition, the term ameliorate or amelioration refers to being capable of reducing the intensity and/or duration of symptoms of a particular disease/condition. Furthermore, the term treat or treatment refers to administration of remedies to a patient after onset of a particular disease/condition.

The term biological substrate in the context of this disclosure refers to the epidermis, dermis, subcutaneous tissues, teeth, gums, smooth and striated muscle tissues, connective tissues, surfaces of internal organs, bone, mucous and other membranes, surfaces of vessels and nerves, cellular surfaces of human and other animals that carry a negative electrostatic charge, and the like.

The term non-biological substrate refers to surfaces of non-biological implants, catheters, indwelling instruments, protheses, fixtures, fastenings, stents, shunts, splints, sponges, gauze pads, bandages, braces, casts, medical implements or devices inserted into or attached to any part of a human or other animal, and other non-biological substrates carrying a negative electrostatic charge, and the like.

Various exemplary embodiments of compositions according to the present invention include cationic surface active transfer agents, hydrophobic barrier-forming materials, carriers and active agents such as dental whitening agents, insect repellant agents and sun protection agents. When exemplary compositions according to the present invention are applied to the surfaces of a biological or non-biological substrate, even wet surfaces of the substrate, the compositions form a coating on the surface, in which the transfer agent binds electrostatically to the surface, the barrier-forming material binds to the transfer agent, and the active agent (e.g., dental whitening agent, insect repellant agent and/or sun protection agent) is available to act on the surface and/or penetrate the surface.

Various exemplary embodiments of methods of prevention, amelioration and/or treatment of a wide variety of diseases and other conditions according to the present invention include applying effective amounts of the compositions according to the present invention to the substrate.

When the compositions according to the present invention are applied to the surfaces of a biological substrate to, for example, prevent, ameliorate and/or treat a disease and/or condition, the recipient of the composition is understood as being a subject in need thereof.

Various exemplary embodiments of composites according to the present invention include compositions according to the present invention provided on applicators.

Various exemplary embodiments of prevention, amelioration and/or treatment of a wide variety of diseases and other conditions according to the present invention include contacting effective amounts of composites according to the present invention with the substrates.

Various exemplary embodiments of compositions according to the present invention include a cationic surface active transfer agent, a hydrophobic barrier-forming material, and an active agent provided on a carrier. When exemplary compositions according to the present invention are applied to a surface of a substrate, even wet surfaces of the substrate, the compositions form a coating on the surface, in which the transfer agent binds electrostatically to the surface, the barrier-forming material binds to the transfer agent, and the active agent is available to act on the surface and/or penetrate the surface.

Various exemplary embodiments of methods of preparing compositions according to the present invention include melting a hydrophobic barrier-forming material, mixing a cationic surface active transfer agent and an active agent carried on a carrier into the molten barrier material, and allowing the combined barrier material, transfer agent and active agent carried on a carrier to solidify.

Various exemplary embodiments of methods of preparing compositions according to the present invention include melting a hydrophobic barrier-forming material, mixing a cationic surface active transfer agent into the molten barrier material, allowing the molten, combined barrier material and transfer agent to solidify, melting the solid, combined barrier material and transfer agent, mixing an active agent carried on a carrier into the molten, combined barrier material and transfer agent, and allowing the combined barrier-forming material, transfer agent and active agent to solidify.

### DETAILED DESCRIPTION OF THE PREFERRED EMBODIMENTS

In various exemplary embodiments, the present invention is directed to compositions including a transfer agent, a barrier material, a carrier, and an active agent. The active agent may be provided on the carrier, which, in turn, is incorporated into the composition.

Varying relative concentrations of components provides control of the rate of elution or delivery of active agents to substrates in relation to or compared with duration of delivery of such active agents.

### Transfer Agent Function

To adhere a hydrophobic barrier material to a wet, hydrophilic, negatively charged surface, a bi-functional transfer agent material is employed. This material has some active groups which are electrostatically positively charged and some active groups which are compatible with and adherent to the hydrophobic components of the barrier material.

Useful transfer agent materials include various primary, secondary, tertiary and cyclic amines, cetyl amine compounds, various diamines (including for example, Duomeens and Ethoduomeens), nitroparaffin-derived heterocyclic amines, and quaternary ammonium compounds. Also useful are compounds of certain cationic polyelectrolytes, invented for the purposes of the present invention and introduced herewith, including, for example, polyethyleneimine (PEI) derivatized with varying concentrations of fatty acids such as, for example, stearic acid, palmitic acid, oleic acid, etc.

Certain of these transfer agents also inhibit the attachment or otherwise defeat the propagation, growth or colonization of bacteria such as, for example, Streptococcus mutans and Streptococcus sobrinus, when added in appropriate concentrations so as to be able to function as a transfer agent and also perform the active agent function.

In various exemplary embodiments, cationic surface active transfer agents are employed in amounts ranging from about 1 to about 40 weight percent, or from 1 to 40 weight percent, based on a total weight of the composition. In further exemplary embodiments, cationic surface active transfer agents are employed in amounts ranging from about 1 to about 10 weight percent, or from 1 to 10 weight percent, based on a total weight of the composition. In still further exemplary embodiments, cationic surface active transfer agents are employed in amounts ranging from about 2 to about 5 weight percent, or from 2 to 5 weight percent, based on a total weight of the composition.

### Transfer Agent Materials

Cationic transfer agent materials useful in the present invention are believed to attach to the desired substrate via a complexing interaction. Said complexing interaction, when described with respect to tooth or skin surfaces, can be said to be between the cationic portion of the material and the proteinaceous portion of the tooth or skin and thus predisposes or conditions the surface of the tooth or skin so that a waxy material will then adhere to the surface. Surface active materials that are capable of strong bonding to the negatively charged and hydrophilic surfaces of both biological and non-biological substrates include various straight-chain alkylammonium compounds, cyclic alkylammonium compounds, petroleum derived cationics, and polymeric cationic materials as described below.

a) Straight-Chain Alkylammonium Compounds

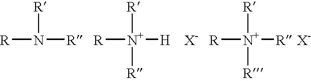

R represents a long (C₈₋₂₀) alkyl chain which may be substituted with one or more hydroxy groups, R′, R″, and R′″ each independently may be either a long (C₈₋₂₀) alkyl chain which may be substituted with one or more hydroxy groups or a smaller (C₁₋₄) alkyl groups which may be substituted with one or more hydroxy groups or aryl (C₆₋₁₀) groups or hydrogen, and X⁻ represents an anion such as chloride or fluoride. These schematic formulas are given for the purpose of defining the classes of compounds and represent the simplest concepts of cationic transfer agents whereby one or more hydrophobic alkyl groups are linked to a cationic nitrogen atom. In many instances the linkage is more complex as, for example, in RCONHCH₂CH₂CH₂N(CH₃)₂. In addition, cationic transfer agents may contain more than one cationic nitrogen atom such as the following class of compounds RNHCH₂ CH₂CH₂NH₂ and derivatives thereof.

Representative examples of compounds according to the above formulas are:

cetyl trimethylammonium chloride (CTAB),

hexadecyltrimethylammonium bromide (HDTAB),

stearyl dimethylbenzylammonium chloride,

lauryl dimethylbenzylammonium chloride,

cetyl dimethylethylammonium halide,

cetyl dimethylbenzylammonium halide,

cetyl trimethylammonium halide,

dodecyl ethyldimethylammonium halide,

lauryl trimethylammonium halide,

coconut alkyltrimethylammonium halide,

N,N—C₈₋₂₀-dialkyldimethylammonium halide, and

specifically compounds such as bis(hydrogenated tallow alkyl) dimethylammonium chloride which is known to adsorb onto the surface with hydrophobic groups oriented away from it, 2-hydroxydodecyl-2-hydroxyethyl dimethyl ammonium chloride and N-octadecyl-N,N′,N′-tris-(2-hydroxyethyl)-1,3-diaminopropane dihydrofluoride.

b) Cyclic Alkylammonium Compounds

A further preferred group of compounds of the present invention which have been found to be applicable includes a class of surface-active quaternary ammonium compounds in which the nitrogen atom carrying the cationic charge is part of a heterocyclic ring. Suitable compounds, for example, are as follows:

laurylpyridinium chloride or bromide,

tetradecylpyridinium bromide,

cetylpyridinium halide (chloride, bromide or fluoride).

c) Petroleum Derived Cationics

Typical basic amines are derived from petroleum-based raw materials such as olefins, paraffins, and aromatic hydrocarbons and include compounds with at least one aliphatic carbon chain containing six or more carbon atoms attached to the nitrogen. Thus, amine salts, diamines, amidoamines, alkoxylated amines, and their respective quaternary salts are applicable.

Preferred compounds of this type include tallow or coco alkyl substituted 1,3-propylene diamines sold by Witco under the trade names of “Adogen” and “Emcol” and similar diamines sold by Akzo under the trade name “Duomeen” and their polyethenoxy derivatives under the trade names of “Ethomeen” and “Ethoduomeens”.

d) Polymeric Amines

Suitable polymeric amines comprise a class of polymers containing ionic groups along the backbone chain and exhibit properties of both electrolytes and polymers. These materials contain nitrogen, of primary, secondary, tertiary or quaternary functionality in their backbone and may have weight average molecular weights as low as about 100 or higher than about 100,000. Representative of these polymeric cationic transfer agents are the following:

polydimeryl polyamine (General Mills Chemical Co.),

polyamide (trade name “Versamide”),

polyacrylamides,

polydiallyldimethylammonium chloride (“Cat-Floc”),

polyhexamethylene biguanide compounds as sold under the trade name “Vantocil”, and also other biguanides, for example those disclosed in U.S. Pat. Nos. 2,684,924, 2,990,425, 3,183,230, 3,468,898, 4,022,834, 4,053,636 and 4,198,425 (all of which are herein incorporated by reference),

1,5-dimethyl-1,5-diazaundecamethylene polymethobromide (“Polybrene” from Aldrich),

polyvinylpyrrolidone and their derivatives,

polypeptides,

poly(allylamine) hydrochloride,

polyoxyethylenated amines, and specifically,

polyethyleneimine (“Polymin” from BASF),

and a class of related and surface active cationic polymers prepared by converting a fraction of the amino groups to their acyl derivatives. The polyethyleneimine is first condensed with less than the stoichiometric quantity of acid halides thus alkylating some of the amino groups and the remaining amino groups are then condensed with hydrogen halides such as hydrogen chloride or, preferentially, hydrogen fluoride. The surface activity of these compounds vary with the number of amino groups which are acylated, and with the chain length of the acylating group RCO——. The condensation reaction is typically performed with stearic or oleic acid chlorides in the presence of a solvent containing metal fluoride, preferentially silver fluoride, in such a manner that silver chloride formed in the reaction precipitates from the solvent.

Also suitable, for the purpose of this invention, are cationic derivatives of polysaccharides such as dextran, starch or cellulose, for example, diethylaminoethyl cellulose (“DEAE-cellulose”). Examples of applicable copolymers based on acrylamide and a cationic monomer are available commercially under the trade name RETEN from Hercules Inc., or under the name FLOC AID from National Adhesives. Particular examples of such polymers are FLOC AID 305 and RETEN 220. Similarly useful are acrylamide-based polyelectrolytes as sold by Allied Colloids under the trade name PERCOL. Further examples of suitable materials are the cationic guar derivatives such as those sold under the trade name JAGUAR by Celanese-Hall.

A further preferred group of compounds which comprises a class of water-insoluble polymers, having nitrogen atoms in their molecules, are quaternary polymers of quaternary ammonium type, betaine type, pyridylpyridinium type or vinylpyridinium-type. Examples are as follows;

poly(vinyl-benzylmethyllaurylammonium chloride),

poly(vinyl-benzylstearylbetaine),

poly(vinyl-benzyllaurylpyridylpyridinium chloride),

poly(vinyl-benzylcetylammonylhexyl ether) and

quaternized polyoxyethyleneated long chain amines, with the general formula RN(CH₃)[(C₂H₄O)ₓH]₂ (+) A(−), where A(−) is generally chloride or fluoride, x is a number from 1 to 20, and R is C₈₋₂₂-alkyl.

These cationic materials, by reacting with the surfaces of the substrate produce strongly hydrophobic films onto which hydrophobic barrier materials are easily transferred by rubbing, smearing, or burnishing.

It is important that the reason for this transferability be understood. The surfaces of the biological or non-biological substrates can be hydrophilic, negatively charged, and potentially “lubricated” by a hydrated biofilm composed of bacteria and other bioorganisms (e.g., surface of teeth). The transfer and adhesion of the barrier materials onto such surfaces is difficult or practically impossible unless the biofilm is modified by a material that is hydrophobic and therefore compatible with the barrier materials.

In a preferred embodiment, the transfer agent, a cationic surfactant, is a polymer which contains a nitrogen atom in a repeating unit and in which a portion of the nitrogen atoms are quaternized by formation of a salt with a C₈₋₂₀ fatty acid, preferably a C₁₂₋₂₀ fatty acid. Examples of such polymeric cationic surfactants include polyacrylamides, polyvinylpyridines, or polyamines, e.g., poly(ethyleneimine), in which from 5 to 95 mole %, preferably 40 to 60 mole %, of the nitrogen atoms have been quaternized by formation of a salt with a fatty acid. Typically such polymers will have a weight average molecular weight of 600 to 60,000, preferably 600 to 1,800.

In a particularly preferred embodiment, the cationic surfactant is a polymer which contains a nitrogen atom in a repeating unit and in which a first portion of the nitrogen atoms are quaternized with a C₈₋₂₀ fatty acid, preferably a C₁₂₋₂₀ fatty acid, and a second portion of the nitrogen atoms are quaternized by forming a salt with HF. Examples of such polymeric cationic surfactants include polyacrylamides, polyvinylpyridines or polyamines, e.g., poly(ethyleneimine), in which from 5 to 95 mole %, preferably from 40 to 60 mole %, of the nitrogen atoms are converted to their acid derivatives by reaction with stearic or oleic acid chlorides, and from 5 to 95 mole %, preferably from 40 to 60 mole %, of the nitrogen atoms are quaternized with HF. In this case, the polymeric cationic surfactant will have a weight average molecular weight of 600 to 60,000, preferably 600 to 1,800.

In another preferred embodiment, the cationic surf actant is a C₈₋₂₀-alkylamine which has been quaternized with HF, such as cetylamine hydrofluoride.

Suitable transfer agents may also include hexahydropyrimidines, such as 5-amino-1,3-bis(2-ethylhexyl)-5-methyl hexahydropyrimidine, which is sold under the trade name hexetidine.

### Barrier Function

Now having a mechanism for adhering a protective, hydrophobic material to the hydrophilic substrate, any of several barrier materials may be selected to perform this function. A microcrystalline wax, for example, is a component in a barrier composition which provides an adherent, conformal, hydrophobic, continuous, inert, colorless or near-colorless barrier which, on application and with subsequent smearing or disturbance, is forced into pits, fissures, cracks and other irregularities of substrate surfaces, thus blocking those sites most vulnerable to occupation by undesirable bacteria and other debris. This waxy barrier appears to endure in place and function indefinitely or until it is mechanically removed. Thus, with the transfer and barrier functions performed, extended protection is provided against deleterious activities since the treated substrate surfaces are entirely sealed against bacteria, acids, staining materials, loss of dental fluorine, etc.

In use, the barrier material is rubbed, on application and thereafter, into pits, cracks, concavities and other depressions. Importantly, barrier materials are amorphous materials which shear or cleave easily so that materials which may adhere to the surface of the barrier may be removed easily by the application of moderate shear forces such as are applied by rubbing, washing, natural exfoliation, the action of the tongue against dental surfaces, toothbrushing, dental flossing, forced water cleaning or vigorous mouth rinsing. This same low-shear characteristic moves the barrier materials about, exposing any active agent substances blended into the barrier materials.

In various exemplary embodiments, hydrophobic barrier-forming materials are employed in amounts ranging from about 40 to about 98 weight percent, or from 40 to 98 weight percent, based on a total weight of the composition. In further exemplary embodiments, hydrophobic barrier-forming materials are employed in amounts ranging from about 70 to about 98 weight percent, or from 70 to 98 weight percent, based on a total weight of the composition. In still further exemplary embodiments, hydrophobic barrier-forming materials are employed in amounts ranging from about 85 to about 93 weight percent, or from 85 to 93 weight percent, based on a total weight of the composition.

### Hydrophobic Barrier Materials

It has been found that various hydrophobic compounds of high molecular weight, solid at body temperature and generally similar to fats and oils are useful as barrier forming materials. Typically they are long chain hydrocarbons, especially normal paraffins having a chain length of 16 carbons or greater, paraffins with several loci of branching and unsaturation, where the extent of such branching and unsaturation does not create unacceptable toxicity nor lower the solidification point below body temperature, and show essentially no solubility in water or saliva. The major types of these wax-like materials belong to two basic categories:

I. Natural waxes of animal, vegetable or mineral origin such as beeswax, lanolin, spermaceti, carnauba wax, petroleum waxes including paraffin waxes and microcrystalline petrolatum; and

II. Synthetic materials including ethylenic polymers such as “Carbowax”, polymethylene wax (“Paraflint”) and various hydrocarbon types as obtained via Fisher-Tropsch synthesis.

Other suitable materials include silicone-based polymers such as polymethylalkylsiloxane, polydimethylsiloxane, poly(perfluoroalkylmethyl siloxane), poly(methyl-3,3,3-trifluoropropyl siloxane) and various aromatic (phenyl containing) siloxanes as sold by Petrarch, which is now United Chemical.

Also useful are various fluoropolymers where some or all of the hydrogen is replaced by fluorine, including, among others: polytetrafluoroethylene (PTFE); fluorinated polyethylene-propylene (FEP); polyvinylidene fluoride (PVDF); and polyvinylfluoride (PVF).

### Active Agent Function

Experimentation with the technology of the present invention demonstrates that active agents may be incorporated into the compositions of the present invention to, e.g., prevent, ameliorate and/or treat a wide variety of diseases and other conditions. For example, active agents may be incorporated into the compositions of the present invention to perform functions such as inhibiting or defeating the attachment and/or propagation, growth or colonization of bacteria on substrate surfaces (e.g., antibacterial functions). In addition, active agents may be incorporated into the compositions of the present invention to perform cosmetic functions, for example whitening of teeth. Active agents may also be incorporated into the compositions of the present invention to provide for protection from the deleterious effects of the sun's rays or artificial light, both visible and invisible. Moreover, active agents may be incorporated into the compositions of the present invention to provide for protection from insects.

Also, active agents incorporated into the compositions of the present invention may provide for any one or more of the following effects: heating; cooling; excitation or procurement of circulation; reduction or prevention of circulation; protection against the attachment, colonization or other activity of biological agents, bacteria, toxins, contaminants, and/or debris; protection against and prevention of adhesion to other cells, tissues, biological structures, non-biological structures, and/or other materials; procurement or stimulation of electrical conductivity or resistivity; protection against UVA, UVB, UVC, and/or other forms and frequencies of radiation; moisturization; hydration; softening; hardening; conveyance of a fragrance; inhibition of a smell; conveyance, prevention, or disguise of a color; deterrence, attraction, or protection against insects, insect bites, parasites, infestations of parasites, and/or microbial infections; enhancement or suppression of the sense of touch; enhancement or suppression of awareness of changes in temperature, pressure, and/or pH; conveyance of a bleaching or whitening effect; protection against changes in color, staining, shrinkage, and/or dehydration; protection or insulation against heat and/or cold; protection against abrasion and/or debridement; protection against hair loss and/or hair growth; protection against pain; enhancement or excitation of intramuscular and/or intraneural communication; reduction or prevention of intramuscular and/or intraneural communication; reduction or prevention of pathological processes, for example inflammation; and/or prevention or reduction of superficial epidermal wrinkles.

Active agents migrate out or diffuse away from the areas on which the compositions according to the present invention are applied so that, to some extent, the effect of the active agents extends to areas not reached by the compositions themselves (e.g., delivery of the active agents to locations other than the surface to which the composition was applied).

Active agents may be blended into the barrier material (when carried on a carrier) so that, as the barrier material is sheared, cleaved, disturbed, eroded, abraded, etc., fresh active agent is exposed and freed to function.

In various exemplary embodiments, active agents are present on the carriers in an amount of from about 1 to about 35 weight percent based on a total weight of the carrier. In further embodiments in which active agents are carried on carriers, active agents are present on the carriers in an amount of from 1 to 35 weight percent based on a total weight of the carrier.

### Active Agent Materials

As indicated above, the active agent is provided on a carrier, which, in turn, is incorporated into the compositions of the present invention. The active agent may include, for example, dental whitening agents, skin protectants and/or sunblocks, insect repellants, active agents imparting an antibacterial function and/or cosmetic function, etc. Any suitable dental whitening agents, skin protectants and/or sunblocks, insect repellants, antibacterial and/or cosmetic agents may be employed.

In various exemplary embodiments, dental whitening agents may include solid dental whitening agents, such as such as peroxides, percarbonates, metal chlorites, persulfates and perborates. Exemplary peroxides include hydrogen peroxide, urea peroxide, calcium peroxide, magnesium peroxide, and mixtures thereof. Exemplary percarbonates include sodium percarbonate and lithium percarbonate. Exemplary metal chlorites include barium chlorite, magnesium chlorite, lithium chlorite and sodium chlorite. Exemplary persulfates include ammonium persulfate. Exemplary perborates include sodium perborate.

Exemplary skin protectants and sun blocks include opaque compounds such as zinc oxide, talc, and other mineral substances. Other protectants/sun blocks include materials that absorb or reflect the sun's rays or the rays of artificial lights such as para-amino benzoic acid, zinc oxide, oxybenzone, avobenzone, cinoxate, dioxybenzone, homosalate, menthyl anthranilate, octocrylene, octyl methoxycinnamate, octyl salicylate, padimate O, phenylbenzimidazole sulfonic acid, sulisobenzone, titanium dioxide, trolamine salicylate, octinoxate, octisalate and the like.

Exemplary insect repellants include DEET, citronella, permethrin, etc.

Among active agents performing an antibacterial function are various cetyl amines, nitroparaffin derivatives, duomeens, ethoxylated duomeens, and other quaternary ammonium compounds. Especially useful is 5-Amino-1,3-bis(2-ethylhexyl)-5-methylhexahydropyrimidine sold under the tradename hexetidine.

Among active agents performing a cosmetic function are peroxides, percarbonates, metal chlorites, persulfates and perborates.

Additionally, suitable active agents may include antibacterial compositions. Examples of such antimicrobial agents belong to the following classes:

(a) Amine-free compounds:
(1) halogenated salicylanilides, as described in U.S. Pat. No. 5,344,641 (herein incorporated by reference), including 4′,5-dibromosalicylanilide, 3,4′,5-trichlorosalicylanilide, 3,4′,5-tribromosalicylanilide, 2,3,3′,5-tetrachlorosalicylanilide, 3,3,3′,5-tetrachlorosalicylanilide, 3,5-dibromo-3′-trifluoromethyl salicylanilide, 5-n-octanoyl-3′-trifluoromethyl salicylanilide, 3,5-dibromo-4′-trifluoromethyl salicylanilide, and 3,5-dibromo-3′-trifluoromethyl salicylanilide (fluorophene);
(2) benzoic esters such as methyl, ethyl, propyl and butyl p-hydroxybenzoic ester;
(3) halogenated diphenyl ethers, as described in U.S. Pat. No. 5,344,641 (herein incorporated by reference), including 2′,4,4′-trichloro-2-hydroxy-diphenyl ether (Triclosan) and 2,2′-dihydroxy-5,5′-dibromo-diphenyl ether;
(4) halogenated carbanilides, as described in U.S. Pat. No. 5,344,641 (herein incorporated by reference), including 3,4,4′-trichlorocarbanilide, 3-trifluoromethyl-4,4′-dichlorocarbanilide and 3,3,4′-trichlorocarbanilide;
(5) phenolic compounds, representative examples of which are described in U.S. Pat. No. 5,290,541 (herein incorporated by reference), which includes phenol, its derivatives and bisphenolic compounds. More specifically, the phenolic compounds (5) include bisphenol A, 2,2′-methylene bis(4-chlorophenol), 2,2′-methylene bis(3,4,6-trichlorophenol) (hexachlorophene), 2,2′-methylene bis(4-chloro-6-bromophenol), bis(2-hydroxy-3,5-dichlorophenyl)sulfide and bis(2-hydroxy-5-chlorobenzyl)sulfide, as well as those listed in the following tables:

Phenol and its Homologs

| | |
|---|---|
| Phenol | -Phenol |
| 2 Methyl | -Phenol |
| 3 Methyl | -Phenol |
| 4 Methyl | -Phenol |
| 4 Ethyl | -Phenol |
| 2,4-Dimethyl | -Phenol |
| 2,5-Dimethyl | -Phenol |
| 3,4-Dimethyl | -Phenol |
| 2,6-Dimethyl | -Phenol |
| 4-n-Propyl | -Phenol |
| 4-n-Butyl | -Phenol |
| 4-n-Amyl | -Phenol |
| 4-tert-Amyl | -Phenol |
| 4-n-Hexyl | -Phenol |
| 4-n-Heptyl | -Phenol |
| 2-Methoxy-4-(2-Propenyl)- | |
| Phenol (Eugenol) | |
| 2-Isopropyl-5-Methyl- | |
| Phenol (Thymol) | |

Mono- and Poly-Alkyl and Aralkyl Halophenols

| | |
|---|---|
| Methyl | -p-Chlorophenol |
| Ethyl | -p-Chlorophenol |
| n-Propyl | -p-Chlorophenol |
| n-Butyl | -p-Chlorophenol |
| n-Amyl | -p-Chlorophenol |
| sec-Amyl | -p-Chlorophenol |
| n-Hexyl | -p-Chlorophenol |
| Cyclohexyl | -p-Chlorophenol |
| n-Heptyl | -p-Chlorophenol |
| n-Octyl | -p-Chlorophenol |
| O-Chlorophenol | |
| Methyl | -o-Chlorophenol |
| Ethyl | -o-Chlorophenol |
| n-Propyl | -o-Chlorophenol |
| n-Butyl | -o-Chlorophenol |
| n-Amyl | -o-Chlorophenol |
| tert-Amyl | -o-Chlorophenol |
| n-Hexyl | -o-Chlorophenol |
| n-Heptyl | -o-Chlorophenol |
| p-Chlorophenol | |
| o-Benzyl | -p-Chlorophenol |
| o-Benzyl-m-methyl | -p-Chlorophenol |
| o-Benzyl-m,m-dimethyl | -p-Chlorophenol |
| o-Phenylethyl | -p-Chlorophenol |
| o-Phenylethyl-m-methyl | -p-Chlorophenol |
| 3-Methyl | -p-Chlorophenol |
| 3,5-Dimethyl | -p-Chlorophenol |
| 6-Ethyl-3-methyl | -p-Chlorophenol |
| 6-n-Propyl-3-methyl | -p-Chlorophenol |
| 6-iso-Propyl-3-methyl | -p-Chlorophenol |
| 2-Ethyl-3,5-dimethyl | -p-Chlorophenol |
| 6-sec-Butyl-3-methyl | -p-Chlorophenol |
| 2-iso-Propyl-3,5-dimethyl | -p-Chlorophenol |
| 6-Diethylmethyl-3-methyl | -p-Chlorophenol |
| 6-iso-Propyl-2-ethyl-3-methyl | -p-Chlorophenol |
| 2-sec-Amyl-3,5-dimethyl | -p-Chlorophenol |
| 2-Diethylmethyl-3,5-dimethyl | -p-Chlorophenol |
| 6-sec-Octyl-3-methyl | -p-Chlorophenol |
| p-Bromophenol | |
| Methyl | -p-Bromophenol |
| Ethyl | -p-Bromophenol |
| n-Propyl | -p-Bromophenol |
| n-Butyl | -p-Bromophenol |
| n-Amyl | -p-Bromophenol |
| sec-Amyl | -p-Bromophenol |
| n-Hexyl | -p-Bromophenol |
| cyclohexyl | -p-Bromophenol |
| o-Bromophenol | o-Bromophenol |
| tert-Amyl | o-Bromophenol |
| n-Hexyl | o-Bromophenol |
| n-Propyl-m,m-dimethyl | |
| 2-Phenyl phenol | |
| 4-Chloro-2-methyl phenol | |
| 4-Chloro-3-methyl phenol | |
| 4-Chloro-3,5-dimethyl phenol | |
| 2,4-Dichloro-3,5-dimethyl phenol | |
| 3,4,5,6-Tetrabromo-2-methylphenol | |
| 5-Methyl-2-pentylphenol | |
| 4-Isopropyl-3-methylphenol | |
| 5-Chloro-2-hydroxydiphenyl | |
| methane | |
| Resorcinol and its Derivatives | |
| Resorcinol | |
| Methyl | -Resorcinol |
| Ethyl | -Resorcinol |
| n-Propyl | -Resorcinol |
| n-Butyl | -Resorcinol |
| n-Amyl | -Resorcinol |
| n-Hexyl | -Resorcinol |
| n-Heptyl | -Resorcinol |
| n-Octyl | -Resorcinol |
| n-Nonyl | -Resorcinol |
| Phenyl | -Resorcinol |
| Benzyl | -Resorcinol |
| Phenylethyl | -Resorcinol |
| Phenylpropyl | -Resorcinol |
| p-Chlorobenzyl | -Resorcinol |
| 5-Chloro | -2,4-Dihydroxydiphenyl Methane |
| 4′-Chloro | -2-4-Dihydroxydiphenyl Methane |
| 5-Bromo | -2,4-Dihydroxydiphenyl Methane |
| 4′-Bromo | -2,4-Dihydroxydiphenyl Methane |

(b) Amine-containing compounds:
(1) antibacterial quaternary ammonium compounds such as alkyldimethylbenzylammonium chloride benzethonium chloride (Hyamine 1622), diisobutylphenoxyethoxyethyl dimethyl benzyl ammonium chloride, and cetyl pyridinium halides (chloride, bromide, iodide, and fluoride). Other materials of this nature are also mentioned in: U.S. Pat. Nos. 2,984,639, 3,325,402, 3,431,208, and 3,703,583, and British Pat. No. 1,319,396 (all of which are herein incorporated by reference).
(2) Further analogous compounds include those in which one or two of the substituents on the quaternary nitrogen has a carbon chain length (typically alkyl group) of some 8 to 20, typically 10 to 18, carbon atoms while the remaining substituents have a lower number of carbon atoms (typically alkyl or benzyl group), such as 1 to 7 carbon atoms, typically methyl or ethyl groups. Typical examples are: dodecyl trimethyl ammonium bromide, and benzyl dimethyl stearyl ammonium chloride.

Preferred antimicrobial materials useful in the present invention belong to the nitroparaffia-in-derived heterocyclic class of compounds. Examples of such compounds may be classified into the following types: monocyclic oxazolidines, bicyclic oxalidines, polymeric bicyclic oxalidines, 1,3-dioxanes, oxazolines, oxazolidinones, and hexahydropyrimidines (e.g., 5-amino-1,3-bis(2-ethylhexyl)-5-methyl hexahydropyrimidine which is sold under the trade name hexetidine).

Other guanine-based antimicrobial substances are: 1,6-bis-(p-chlorophenyldiguanidine)hexane (also known by the trade name “chlorhexidine”), 1,6-di-(2-ethylhexyldiguanidine)hexane (known as “alexidine”), and 1,1′-hexamethylene-bis-{5-(4-fluorophenyl)-diguanidine} (also known as “fluorhexidine”).

Active agents that may be provided on a carrier, which, in turn, is incorporated into compositions according to the present invention may also include a source of fluoride, such as sodium fluoride, potassium fluoride, tin fluoride, zinc fluoride, organic fluorides such as long-chained aminofluorides, for example oleylaminofluoride, cetyl aminofluoride or ethanolaminohydrofluoride, fluorosilicates, for example, potassium hexafluorosilicate or sodium hexafluorosilicate, fluorophosphates such as ammonium, sodium, potassium, magnesium or calcium fluorophosphate and/or fluorozirconates, for example sodium, potassium or tin fluorozirconate.

Active agents that may be provided on a carrier, which, in turn, is incorporated into compositions according to the present invention may also include one or more antibiotics, such as penicillin, polymyxin B, vancomycin, kanamycin, erythromycin, niddamycin, metronidazole, spiramycin and tetracycline.

Active agents that may be provided on a carrier, which, in turn, is incorporated into compositions according to the present invention may also include skin treatments, such as Lanacane, cortizone, cayenne, capsicum, Retin-A and shark liver oil and sun protection agents, such as zinc oxide, talc, oxybenzone, para-aminobenzoic acid, octinoxate and octisalate.

### Carriers

As indicated above, the active agent is provided on a carrier, which, in turn, is incorporated into compositions according to the present invention. “Providing” the active agent on the carrier is intended to encompass any suitable mechanism by which an active agent can be carried by a carrier. As discussed below, the carrier may be a porous carrier. Accordingly, an active agent provided on a carrier is intended to encompass an arrangement whereby at least a portion of the active agent is provided within the pores of the carrier. An active agent provided on a carrier may also encompass an arrangement whereby the active agent is fully incorporated into the carrier. Further, an active agent provided on a carrier is intended to encompass an arrangement whereby at least a portion of the active agent is adsorbed to the surface of, e.g., a nonporous carrier. An active agent provided on a carrier may also encompass an arrangement whereby the entirety of the active agent is adsorbed to the surface of, e.g., a nonporous carrier.

The carriers that may be employed in the compositions according to the present invention are not particularly limited. In various exemplary embodiments, carriers employed in the compositions according to the present invention may include inorganic porous or nonporous carriers. Exemplary inorganic porous particles include porous silica gels. Exemplary silica gels include Davisil, grade 643, which has a particle size of from 35 to 70 μm and a water absorbency of 1.15 cc/g. Further exemplary silica gels include Silcron G100 gel particles, manufactured by Millennium Chemicals, which have a particle size of from 5 to 8 μm with water absorbency of 1.5 cc/g. Exemplary inorganic nonporous particles include fumed silicas. Exemplary fumed silicas include CAB-O-SIL fumed silica, which has a particle size of about 40 um (agglomerants of 0.2 to 0.3 μm particles) and a water absorbency of about 1.5 cc/g.

Exemplary carriers may include porous particles with a high internal volume capable of absorbing or incorporating various liquids (e.g., liquid active agents and/or dissolved solid active agents). Silica gel particles such as Silcron function according to this principle. There is some adsorption of active agents onto the surface of such particles but most of the active agent is carried within the particle body. By contrast, carriers such as CAB-O-SIL powder include fumed solid and non-porous silica particles having surfaces to which active agents may be adsorbed.

The carriers that may be employed in the compositions according to the present invention preferably have a mean particle size of from 1 μm to 100 μm, more preferably from 1 μm to 10 μm for non-porous carriers and from 5 μm to 40 μm for porous carriers.

The carriers that may be employed in the compositions according to the present invention preferably have a water absorbency between 1 and 10 cc/g.

The carriers that may be employed in the compositions according to the present invention preferably have an oil absorbency between 1 and 12g/g.

The carriers that may be employed in the compositions according to the present invention, with active agent(s) carried thereon, preferably account for 20 and 50% by volume of the composition.

In various exemplary embodiments, carriers that may be employed in the compositions according to the present invention may be surface modified. Inorganic particles, such as the silica gel particles described above, typically have a very large surface area (e.g., from 1000 to 3000 ft²/g) and thus, e.g., through adsorption, can deplete the compositions of transfer agents that are necessary for adhesion of the compositions according to the present invention to negatively charged substrates. It is possible to reduce the effect of this depletion of transfer agents by treating the carriers, which may carry a negative surface charge in aqueous media, to provide a positive surface charge. This provision of a positive surface charge reduces the propensity of carriers to adsorb transfer agents. The provision of a positive surface charge to carriers can be carried out by any suitable method. In various exemplary embodiments, provision of a positive surface charge to carriers is carried out by treating the carriers with metal ions, cationic surfactants and/or transfer agents, prior to loading the carriers with active agents. In embodiments, negatively charged surfaces of carriers are converted to positively charged surfaces by adsorption of aluminum cations from an aqueous solution of aluminum nitrate. As an alternative to aluminum cations, cations of zinc, magnesium, nickel or other metals may be employed.

In addition to surface treating carriers to reduce the effect of depletion of transfer agents caused by the carriers, it is also possible to employ an excess of transfer agent to compensate for any loss of effect cause be adsorption of transfer agent to the carriers.

In addition to the carriers discussed above, carriers that may be employed in the compositions according to the present invention may include porous polymeric particles. Exemplary porous polymeric particles include the Poly-Pore delivery system, manufactured by AMCOL Corp., which includes particles having a median particle size of 40 μm and a water absorbency of 6-8 cc/g.

### Additional Components

In addition to the components described above, additional components may be added to compositions according to the present invention. Such additional components include, for example, stabilizers, viscosity modifiers, colorants, odorants and flavorants. Exemplary stabilizers include various pyrophosphate salts, phosphoric acid and other mineral acids. Exemplary viscosity modifiers include low viscosity hydrocarbon-based oils or waxes. Exemplary odorants and flavorants include natural or synthetic oils such as, for example, peppermint oil, spearmint oil, eucalyptus oil and cinnamon oil. Exemplary colorants include natural or synthetic materials such as, for example carmine and indigo.

### Methods of Delivery of Active Agents

The compositions according to the present invention, and therefore the active agents contained therein, may be delivered to the desired substrate by any suitable method. In various exemplary embodiments, the active agents are delivered from the interior of a film of the composition applied to a substrate to both the substrate surface of the film and the surface opposite to the substrate surface. In addition, the active agents can extend to areas not reached by the compositions themselves (e.g., delivery of the active agents to locations other than the surface to which the composition was applied via penetration). The application of the composition to the substrate having been performed by any suitable method, for example by rubbing, spraying, pouring, swallowing, rinsing, deposition, etc.

Furthermore, it is to be understood that varying relative concentrations of the components of the compositions according to the present invention provide control of the rate of elution or delivery of active agent(s) to the substrate(s) in relation to or compared with the duration of the delivery of such active agent(s).

The compositions according to the present invention are composed of a network of interconnected carrier particles wherein the transfer of the active agents to the substrate occurs by diffusion and can be enhanced by the frequency of interconnected bridges between the particles. The frequency of the interconnected bridges is related to the volumetric fraction of the carrier particles in the composition. The volumetric fraction of the carrier particles in the composition is preferably 20 to 50% of the total volume of the composition. Differing volumetric fractions are possible and depend on the ultimate mode of application of the composition.

The composition according to the present invention to be administered will contain an effective amount of the active agent. Actual methods of preparing such dosage forms are known and will be apparent to those skilled in this art. As used herein, the term “effective amount” refers to that amount which would prevent, ameliorate and/or treat the subject disease/condition. Accordingly, the “effective amount” can be a therapeutically effective amount, useful for treating someone already afflicted with the disease/condition, or can be a prophylactically effective amount useful for prevention and/or amelioration of a disease/condition in a patient. It will be appreciated that the effective amount will vary from patient to patient depending on such factors as the subject disease/condition being, the severity of the disease/condition, the size of the patient being treated, the patient's ability to mount an immune response, and the like. The determination of an effective amount for a given patient is within the skill of one practicing in the art.

### Methods of Preparing Compositions

The compositions according to the present invention may be prepared by any suitable method. In various exemplary embodiments, compositions according to the present invention are prepared by a method in which the barrier material is first melted. The transfer agent is then added and the combined barrier material and transfer agent are solidified, e.g., by allowing the composition to cool. The active agent carried on a carrier may be added at the same time as the transfer agent, or after the barrier material and transfer agent have been combined and solidified. In such cases where the barrier material and transfer agent have been combined and solidified, it may be necessary to melt the combined barrier material and transfer agent before adding the active agent.

The compositions may be applied directly to the surface to be treated, for example skin or teeth, or may be combined with an applicator (e.g., bandaging, dental floss, toothbrush, toothpick, swab, syringe, etc.). In such embodiments, the combined compositions and applicator may be prepared by any suitable means. In various exemplary embodiments, the applicator may be dipped in a precursor to the compositions according to the present invention (e.g., before the compositions are solidified) and then cooled to provide a composite. In further exemplary embodiments, the completed compositions according to the present invention may be remelted, and the applicator dipped in the thus-formed liquid, followed by cooling.

In various exemplary embodiments of the present invention, methods of, for example, preventing, ameliorating and/or treating numerous diseases and conditions of substrates may be carried out by contacting the compositions according to the present invention with the substrate to effect transfer of the compositions to the surface of the substrate. This can be carried out by directly applying the compositions, or by using composites as described above. The exact means of contacting will depend of course on the nature of the delivery system. Thus for dental application for example, in the case of a dental floss, flossing will suffice, while brushing will suffice, in the case of a toothbrush. Rubbing will be appropriate for toothpicks, cotton swabs, bandaging, etc.

### Applications

Exemplary compositions according to the present invention may comprise the certain active agents in order to achieve one or more corresponding effects as follows:

1) A formulation of the composition according to the present invention comprising one or more compounds such as capsaicin, capsicum oleoresin, and the like, as active agent applied to a biological substrate and/or non-biological substrate to enhance circulation in adjacent and nearby tissues.

2) A formulation of the composition according to the present invention comprising one or more antimicrobials such as quaternary ammonium compounds such as cetylpyridinium chloride or iodine, antibiotics such as penicillin or metronidazole, antifungals such as cyclosporins or clortrimazole, and/or other biocides such as 2-(1-methylpropyl)phenyl methylcarbamate or methoprene as active agent applied to a biological substrate and/or non-biological substrate to prevent, reduce or eliminate infection in adjacent and nearby tissues.

3) A formulation of the composition according to the present invention comprising one or more compounds such as cortisone, aspirin, and the like, as active agent applied to a biological substrate and/or non-biological substrate to prevent, reduce or eliminate the deleterious effects of toxic or irritating materials in adjacent and nearby tissues, and/or to prevent, reduce or eliminate inflammation in adjacent and nearby tissues.

4) A formulation of the composition according to the present invention comprising one or more compounds such as vitamin E, mineral oil, and the like, as active agent applied to a biological substrate and/or non-biological substrate to prevent, reduce or eliminate the drying or dehydration of adjacent and nearby tissues, and/or to soften the substrate.

5) A formulation of the composition according to the present invention comprising water as active agent applied to a biological substrate and/or non-biological substrate to hydrate or moisturize adjacent and nearby tissues.

6) A formulation of the composition according to the present invention comprising one or more compounds such as mineral oil, glucosamine, and the like, as active agent applied to a biological substrate and/or non-biological substrate to lubricate adjacent and nearby tissues.

7) A formulation of the composition according to the present invention comprising one or more compounds such as menthol, methyl mercaptan, and the like, as active agent applied to a biological substrate and/or non-biological substrate to impart a fragrance for purposes of identifying the presence or depletion of the formulation with passage of time.

8) A formulation of the composition according to the present invention comprising one or more compounds such as cochineal, ferric oxide, and the like, as active agent applied to a biological substrate and/or non-biological substrate to impart a color for purposes of identifying the presence or depletion of the formulation with passage of time, and/or to impart a color for cosmetic purposes.

9) A formulation of the composition according to the present invention comprising one or more compounds such as polytetrafluoroethylene, microcrystalline wax, and the like, as active agent applied to a biological substrate and/or non-biological substrate to prevent the formation of adhesions of tissue to adjacent and nearby tissues and/or to the non-biological substrate, to protect against staining or discoloration of the substrate, and/or to protect adjacent and nearby surfaces against contamination by debris.

10) A formulation of the composition according to the present invention comprising one or more compounds such as microcrystalline wax, mineral oil, and the like, as active agent applied to a biological substrate and/or non-biological substrate to insulate against the effects of chemical agents.

11) A formulation of the composition according to the present invention comprising one or more compounds such as menthol, cinnamyl anthranilate, and the like, as active agent applied to a biological substrate and/or non-biological substrate to impart a fragrance for cosmetic purposes.

12) A formulation of the composition according to the present invention comprising one or more compounds such as hexetidine, n-butyl acrylate, and the like, as active agent applied to a biological substrate and/or non-biological substrate to enhance the adhesion of the non-biological substrate to the biological substrate.

13) A formulation of the composition according to the present invention comprising one or more compounds such as aluminum hydroxide, zinc chloride, and the like, as active agent applied to a biological substrate and/or non-biological substrate to prevent or reduce bleeding.

14) A formulation of the composition according to the present invention comprising one or more compounds such as warfarin, heparin, and the like, as active agent applied to a biological substrate and/or non-biological substrate to prevent or reduce coagulation.

15) A formulation of the composition according to the present invention comprising one or more compounds such as aspirin, morphine, and the like, as active agent applied to a biological substrate and/or non-biological substrate to prevent or reduce pain.

16) A formulation of the composition according to the present invention comprising one or more compounds such as capsaicin, menthol, and the like, as active agent applied to a biological substrate and/or non-biological substrate to prevent or reduce sensation.

17) A formulation of the composition according to the present invention comprising one or more compounds such as cyclosporins, clotrimazole, and the like, as active agent applied to a biological substrate and/or non-biological substrate to protect or treat adjacent surfaces against fungal infections.

18) A formulation of the composition according to the present invention comprising one or more compounds such as shark liver oil, capsaicin, and the like, as active agent applied to a biological substrate and/or non-biological substrate for the cosmetic reduction of superficial wrinkles.

19) A formulation of the composition according to the present invention comprising one or more compounds such as DEET, citronella oil, eucalyptus, and the like, as active agent applied to a biological substrate and/or non-biological substrate to prevent or reduce insect bites.

The above written description provides a manner and process of making and using the present invention such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and sub-ranges within a numerical limit or range are specifically included as if explicitly written out.

The above description is presented to enable a person skilled in the art to make and use the present invention, and is provided in the context of a particular application and its requirements. Various modifications to the preferred embodiments will be readily apparent to those skilled in the art, and the generic principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Having generally described the present invention, a further understanding can be obtained by reference to certain specific examples, which are provided herein for purposes of illustration only, and are not intended to be limiting unless otherwise specified.

### EXAMPLES

In the following examples, and throughout this specification, all parts and percentages are by weight, and all temperatures are in degrees Celsius, unless expressly stated to be otherwise. Where the solids content of a dispersion or solution is reported, it expresses the weight of solids based on the total weight of the dispersion or solution, respectively. Where a molecular weight is specified, it is the molecular weight range ascribed to the product by the commercial supplier, which is identified. Generally this is believed to be weight average molecular weight.

### Example I

### Surface Treatment of Carrier

10 g of silica powder (Silcron G 100) are added to a 100 ml beaker and combined with 50 ml of a 5% solution of aluminum nitrate. The resulting dispersion is mixed for 60 min and then filtered. After filtration, the filtrate powder is transferred to a 1 liter beaker containing 500 ml of distilled water and the combined components are mixed for 60 min. The mixture is then filtered. The filtrate is dried overnight in a 110° C. oven.

### Example II

### Treatment/Protection Compositions Using Liquid Oxidizers

In the following examples, a liquid dental whitening agent, a 35% solution of hydrogen peroxide, forms from about 5% to about 25% by weight of the prepared treatment/protection compositions. The liquid dental whitening agent is added to a dry powder of silica gel particles and incorporated, by mixing, into a prepared composition including a hydrophobic barrier and a transfer agent. The composition including the hydrophobic barrier and the transfer agent includes, in particular, Witco 835 Multiwax with the viscosity modifier Witco Kaydol, as a hydrophobic barrier, and Hexetidine, as the transfer agent.

### Example II-A

40 g of the prepared composition including a hydrophobic barrier and a transfer agent are added to a beaker and melted at a temperature not exceeding 100° C. 10 g of mineral oil and 5 g of eucalyptus oil are then added to the liquefied formulation and mixed until a homogenous mixture is obtained. The solution is cooled to 60° C.

Separately, 10 g of 35% hydrogen peroxide are added to 3 g of Silcron G100 silica gel particles. After the peroxide is fully absorbed by the silica, the wet silica powder is added to the homogenous mixture described above, and stirred, at a high shear rate, until a homogenous and smooth paste is obtained.

### Example II-B

40 g of the prepared composition including a hydrophobic barrier and a transfer agent are added to a beaker and melted at a temperature not exceeding 100° C. 10 g of mineral oil and 5 g of eucalyptus oil are then added to the liquefied formulation and mixed until a homogenous mixture is obtained. The solution is cooled to 60° C.

Separately, 10 g of 35% hydrogen peroxide stabilized by addition of 10% of citric acid are added to 3 g of Silcron G100 silica gel particles. After the peroxide is fully absorbed by the silica, the wet silica powder is added to the homogenous mixture described above, and stirred, at a high shear rate, until a homogenous and smooth paste is obtained.

### Example II-C

40 g of the prepared composition including a hydrophobic barrier and a transfer agent are added to a beaker and melted at a temperature not exceeding 100° C. 10 g of mineral oil and 5 g of eucalyptus oil are then added to the liquefied formulation and mixed until a homogenous mixture is obtained. The solution is cooled to 60° C.

Separately, 10 g of 35% hydrogen peroxide are added to 3 g of Davisil silica gel particles. After the peroxide is fully absorbed by the silica, the wet silica powder is added to the homogenous mixture described above, and stirred, at a high shear rate, until a homogenous and smooth paste is obtained.

### Example II-D

40 g of the prepared composition including a hydrophobic barrier and a transfer agent are added to a beaker and melted at a temperature not exceeding 100° C. 10 g of mineral oil and 5 g of eucalyptus oil are then added to the liquefied formulation and mixed until a homogenous mixture is obtained. The solution is cooled to 60° C.

Separately, 10 g of 35% hydrogen peroxide are added to 3 g of CAB-O-SIL fumed silica particles. After the peroxide is fully absorbed by the silica, the wet silica powder is added to the homogenous mixture described above, and stirred, at a high shear rate, until a homogenous and smooth paste is obtained.

### Example II-E

40 g of the prepared composition including a hydrophobic barrier and a transfer agent are added to a beaker and melted at a temperature not exceeding 100° C. 10 g of mineral oil and 5 g of eucalyptus oil are then added to the liquefied formulation and mixed until a homogenous mixture is obtained. The solution is cooled to 60° C.

Separately, 20 g of 35% hydrogen peroxide are added to 6 g of Silcron G100 silica gel particles. After the peroxide is fully absorbed by the silica, the wet silica powder is added to the homogenous mixture described above, and stirred, at a high shear rate, until a homogenous and smooth paste is obtained.

### Example III

### Treatment/Protection Composition Using Solid Oxidizers

According to the following example, a solid dental whitening agent, such as urea peroxide, can constitute from about 5% to about 35% by weight of a prepared treatment/protection composition. Suitable urea peroxide may have a particle size of from about 5 to about 100 μm. Urea peroxide may be incorporated into a prepared composition including a hydrophobic barrier and a transfer agent by strong mixing. During mixing a temperature of the formulation may be maintained between 60 and 80° C.

In this particular example, a solid dental whitening agent, urea peroxide, forms 25% by weight of the prepared treatment/protection composition. The urea peroxide is incorporated into the prepared composition including a hydrophobic barrier and a transfer agent by strong mixing.

60 g of the prepared composition including a hydrophobic barrier and a transfer agent are added to a beaker and melted at a temperature not exceeding 100° C. 25 g of mineral oil and 5 g of eucalyptus oil are then added to the liquefied formulation and mixed until a homogenous mixture is obtained. The solution is cooled to 60° C.

30 g of urea peroxide powder (mean particle size of 50 μm) is added to the homogenous mixture described above, and stirred, at a high shear rate, until a homogenous and smooth paste is obtained.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

### Example IV

### Treatment/Protection Compositions Using Permethrin

40 g of the prepared composition including a hydrophobic barrier composition and a transfer agent are added to a beaker and melted at a temperature not exceeding 100° C. The liquefied formulation is mixed until a homogenous mixture is obtained and cooled to approx. 60° C.

Separately, a 20 g permethrin solution (consisting of 36.8% permethrin and 63.2% petroleum distillates) are added to 6 g of Silcron G100 silica gel particles. After the permethrin solution is fully absorbed by the silica, the wet silica carrier is added to the mixture described above and stirred, at a high shear rate, until a homogenous and smooth paste is obtained.

In the above-described formulation, permethrin can be trapped in the porous carrier and the distribution of the permethrin to the desired substrate is enhanced and it's activity is significantly prolonged. Additionally, such formulations spread easily onto the substrate and exhibited superior adherence to wet surfaces of substrates.

### Example V

### Treatment/Protection Compositions Using DEET

DEET is incorporated into the pores, if any present, and onto the surfaces of a carrier. The evaporation rate of DEET from a thin film was then measured by weight. The film was smeared onto a glass slide and its weight was measured as a function of time.

The initial weight of the film was 0.0231 grams and the area that was covered was approximately 3 inches (thickness ca 10 microns). The weight of the film leveled off at 0.019 grams when the DEET was fully evaporated, approximately 36 hours after application. Half of the DEET, about 2 milligrams on 3 square inches, remained 16 hours after application.

### Example V-A

60 g of the prepared composition including a hydrophobic barrier and a transfer agent are added to a beaker and melted at a temperature not exceeding 100° C. 5 g of citronella oil is then added to the liquefied formulation and mixed until a homogenous mixture is obtained. The solution is cooled to 60° C.

Separately, 20 g of DEET oil is added to 6 g of charge-converted Silcron G100 silica gel particles. After the DEET is fully absorbed by the silica, the wet silica powder is added to the homogenous mixture described above, and stirred, at a high shear rate, until a homogenous and smooth paste is obtained.

### Example V-B

60 g of the prepared composition including a hydrophobic barrier and a transfer agent are added to a beaker and melted at a temperature not exceeding 100° C. 5 g of citronella oil is then added to the liquefied formulation and mixed until a homogenous mixture is obtained. The solution is cooled to 60° C.

Separately, 20 g of DEET oil is added to 2 g of Poly-Pore E200. After DEET is fully absorbed by the carrier, the wet powder is added to the homogenous mixture described above, and stirred, at a high shear rate, until a homogenous and smooth paste is obtained. (Poly-Pore E200 is allylmethacrylate crosspolymer manufactured by AMCOL Corp. with a median particle size of 40um, a water absorbency of 6-8 g/g, and an oil absorbency of 9-12 g/g.)

1. A treatment/protection composition, comprising:
a cationic surface active transfer agent;
a hydrophobic barrier-forming material;
an active agent; and
a carrier;
wherein an effective amount of active agent is provided on the surfaces of the carrier.

2. The composition of claim 1, wherein when the composition is applied to a surface of a substrate, the composition forms a coating on the surface, in which the transfer agent binds electrostatically to the surface, the barrier-forming material binds to the transfer agent, and the active agent provided on the carrier is available to act on the surface.

3. The composition of claim 1, wherein the transfer agent comprises at least one member selected from the group consisting of compounds of the formulae (I), (II), and (III): 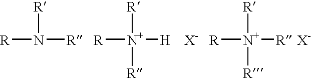
wherein R is a C₈₋₂₀ alkyl chain which may be substituted with one or more hydroxy groups, R′, R″, and R′″ each independently are a C₈₋₂₀ alkyl chain which may be substituted with one or more hydroxy groups, a C₁₋₄ alkyl group which may be substituted with one or more hydroxy groups, a C₆₋₁₀ aryl group, or hydrogen, and X is fluoride or chloride; laurylpyridinium chloride; laurylpyridinium bromide; cetylpyridinium chloride; cetylpyridinium bromide; cetylpyridinium fluoride; petroleum derived cationics; polydimeryl polyamines; polydiallyl dimethylammonium chlorides, polyhexamethylene biguanides; 1,5-dimethyl-1,5-diazaundecamethylene polymethobromide; polyvinylpyrrolidones; polypeptides; poly(allylamine)hydrochloride; polyoxyethylenated amines; acylated polyethyleneimines; diethylaminoethyl cellulose; poly(vinyl-benzylstearylbetaine); poly(vinyl-benzylmethyllaurylammonium chloride); poly(vinyl-benzyllaurylpyridylpyridinium chloride); poly(vinyl-benzylcetylammonylhexyl ether); compounds of the formula RN(CH₃)[(C₂H₄O)ₓH]₂ (+) A(−), where A(−) is chloride or fluoride, X is a number from 1 to 20, and R is a C₈₋₂₂ alkyl group; polyacrylamide in which 5 to 95 mole % of the nitrogen atoms have been derivatized by reaction with a C₈₋₂₀ fatty acid halide and 5 to 95 mole % of the nitrogen atoms have been quaternized with HF; polyvinylpyridine in which 5 to 95 mole % of the nitrogen atoms have been derivatized by reaction with a C₈₋₂₀ fatty acid halide and 5 to 95 mole % of the nitrogen atoms have been quaternized with HF; and 5-amino-1,3-bis(2-ethylhexyl)-5-methylhexahydropyrimidine.

4. The composition of claim 1, wherein the barrier-forming material comprises at least one member selected from the group consisting of beeswax, lanolin, spermaceti, carnauba wax, paraffin waxes, microcrystalline waxes, petrolatum waxes, ethylenic polymer waxes, polymethylene waxes, polymethylalkylsiloxane, polydimethylsiloxane, poly(perfluoroalkylmethyl siloxane), poly(methyl-3,3,3-trifluoropropyl siloxane), polytetrafluoroethylene, fluorinated polyethylene-propylene, polyvinylidene fluoride, and polyvinylfluoride.

5. The composition of claim 1, wherein the transfer agent is present in an amount of from about 1 to about 10 weight percent, based on a total weight of the composition.

6. The composition of claim 1, wherein the barrier-forming material is present in an amount of from about 70 to about 98 weight percent, based on a total weight of the composition.

7. The composition of claim 1, wherein the active agent is present in an amount of from about 1 to about 35 weight percent, based on a total weight of the carrier.

8. The composition of claim 1, wherein the transfer agent is 5-amino-1,3-bis(2-ethylhexyl)-5-methylhexahydropyrimidine.

9. The composition of claim 1, wherein the barrier-forming material is a microcrystalline wax.

10. The composition of claim 1, wherein the active agent is a dental whitening agent.

11. The composition of claim 1, wherein the active agent is an insect repellant.

12. The composition of claim 1, wherein the active agent is a sun protectant.

13. The composition of claim 1, comprising:
5-amino-1,3-bis(2-ethylhexyl)-5-methylhexahydropyrimidine as the transfer agent in an amount of from about 1 to about 10 weight percent, based on a total weight of the composition;
a microcrystalline wax as the barrier-forming material in an amount of from about 70 to about 98 weight percent, based on a total weight of the composition; and
urea peroxide as the dental whitening active agent in an amount of from about 1 to about 20 weight percent, based on a total weight of the composition.

14. The composition of claim 1, wherein the active agent comprises at least one member selected from the group consisting of hydrogen peroxide, urea peroxide, calcium peroxide, magnesium peroxide, sodium percarbonate, lithium percarbonate, barium chlorite, magnesium chlorite, lithium chlorite, sodium chlorite, ammonium persulfate, sodium perborate, 4′5-dibromosalicylanilide, 3,4′5-trichlorosalicylanilide, 3,4′5-tribromosalicylanilide, 2,3,3′,5-tetrachlorosalicylanilide, 3,3,3′5-tetrachlorosalicylanilide, 3,5-dibromo-3′-trifluoromethyl salicylanilide, 5-n-octanoyl-3′-trifluoromethyl salicylanilide, 3,5-dibromo-4′-trifluoromethyl salicylanilide, 3,5-dibromo-3′-trifluoromethyl salicylanilide, methyl p-hydroxybenzoic ester, ethyl p-hydroxybenzoic ester, propyl p-hydroxybenzoic ester, butyl p-hydroxybenzoic ester, 2′4,4′-trichloro-2-hydroxy-diphenyl ether, 2,2′-dihydroxy-5,5′-dibromo-diphenyl ether, 3,4,4′-trichlorocarbanilide, 3-trifluoromethyl-4,4′-dichlorocarbanilide, 3,3,4′-trichlorocarbanilide, phenol, 2-methyl-phenol, 3-methyl-phenol, 4-methyl-phenol, 4-ethyl-phenol, 2,4-dimethyl-phenol, 2,5-dimethyl-phenol, 3,4-dimethyl-phenol, 2,6-dimethyl-phenol, 4-n-propyl-phenol, 4-n-butyl-phenol, 4-n-amyl-phenol, 4-tert-amyl-phenol, 4-n-hexyl-phenol, 4-n-heptyl-phenol, 2-methoxy-4-(2-propenyl)-phenol, 2-isopropyl-5-methyl-phenol, methyl-p-chlorophenol, ethyl-p-chlorophenol, n-propyl-p-chlorophenol, n-butyl-p-chlorophenol, n-amyl-p-chlorophenol, sec-amyl-p-chlorophenol, n-hexyl-p-chlorophenol, cyclohexyl-p-chlorophenol, n-heptyl-p-chlorophenol, n-octyl-p-chlorophenol, methyl-o-chlorophenol, ethyl-o-chlorophenol, n-propyl-o-chlorophenol, n-butyl-o-chlorophenol, n-amyl-o-chlorophenol, tert-amyl-o-chlorophenol, n-hexyl-o-chlorophenol, n-heptyl-o-chlorophenol, o-benzyl-p-chlorophenol, o-benzyl-m-methyl-p-chlorophenol, o-benzyl-m,m-dimethyl-p-chlorophenol, o-phenylethyl-p-chlorophenol, o-phenylethyl-m-methyl-p-chlorophenol, 3-methyl-p-chlorophenol, 3,5-dimethyl-p-chlorophenol, 6-ethyl-3-methyl-p-chlorophenol, 6-n-propyl-3-methyl-p-chlorophenol, 6-iso-propyl-3-methyl-p-chlorophenol, 2-ethyl-3,5-dimethyl-p-chlorophenol, 6-sec-butyl-3-methyl-p-chlorophenol, 2-iso-propyl-3,5-dimethyl-p-chlorophenol, 6-diethylmethyl-3-methyl-p-chlorophenol, 6-iso-propyl-2-ethyl-3-methyl-p-chlorophenol, 2-sec-amyl-3,5-dimethyl-p-chlorophenol, 2-diethylmethyl-3,5-dimethyl-p-chlorophenol, 6-sec-octyl-3-methyl-p-chlorophenol, methyl-p-bromophenol, ethyl-p-bromophenol, n-propyl-p-bromophenol, n-butyl-p-bromophenol, n-amyl-p-bromophenol, sec-amyl-p-bromophenol, n-hexyl-p-bromophenol, cyclohexyl-p-bromophenol, tert-amyl-o-bromophenol, n-hexyl-o-bromophenol, n-propyl-m,m-dimethyl-o-bromophenol, 2-phenylphenol, 4-chloro-2-methylphenol, 4-chloro-3-methylphenol, 4-chloro-3,5-dimethylphenol, 2,4-dichloro-3,5-dimethylphenol, 3,4,5,6-tetrabromo-2-methylphenol, 5-methyl-2-pentylphenol, 4-isopropyl-3-methylphenol, 5-chloro-2-hydroxydiphenylmethane, resorcinol, methyl-resorcinol, ethyl-resorcinol, n-propyl-resorcinol, n-butyl-resorcinol, n-amyl-resorcinol, n-hexyl-resorcinol, n-heptyl-resorcinol, n-octyl-resorcinol, n-nonyl-resorcinol, phenyl-resorcinol, benzyl-resorcinol, phenylethyl-resorcinol, phenylpropyl-resorcinol, p-chlorobenzyl-resorcinol, 5-chloro-2,4-dihydroxydiphenylmethane, 4′-chloro-2,4-dihydroxydiphenylmethane, 5-bromo-2,4-dihydroxydiphenylmethane, 4′-bromo-2,4-dihydroxydiphenylmethane, 2,2′-methylene bis(4-chlorophenol), 2,2′-methylene bis(3,4,6-trichlorophenol), 2,2′-methylene bis(4-chloro-6-bromophenol), bis(2-hydroxy-3,5-dichlorophenyl)sulfide, bis(2-hydroxy-5-chlorobenzyl)sulfide, benzethonium chloride, diisobutylphenoxyethoxyethyl dimethyl benzyl ammonium chloride, cetyl pyridinium chloride, cetyl pyridinium bromide, cetyl pyridinium iodide, cetyl pyridinium fluoride, dodecyl trimethyl ammonium bromide, benzyl dimethyl stearyl ammonium chloride, monocyclic oxazolidines, bicyclic oxalidines, polymeric bicyclic oxalidines, 1,3-dioxanes, oxazolines, oxazolidinones, 5-amino-1,3-bis(2-ethylhexyl)-5-methyl hexahydropyrimidine, 1,6-bis-(p-chlorophenyldiguanidine)hexane, 1,6-di-(2-ethylhexyldiguanidine)hexane, 1,1′-hexamethylene-bis-{5-(4-fluorophenyl)-diguanidine}, sodium fluoride, potassium fluoride, tin fluoride, zinc fluoride, oleylaminofluoride, cetyl aminofluoride, ethanolaminohydrofluoride, potassium hexafluorosilicate, sodium hexafluorosilicate, sodium fluorophosphate, ammonium fluorophosphate, potassium fluorophosphate, magnesium fluorphosphate, calcium fluorophosphate, sodium fluorozirconate, potassium fluorozirconate, tin fluorozirconate, penicillin, polymyxin B, vancomycin, kanamycin, erythromycin, niddamycin, metronidazole, spiramycin, tetracycline, Lanacane, cortizone, cayenne, capsicum, Retin-A, shark liver oil, zinc oxide, talc, oxybenzone, para-aminobenzoic acid, octinoxate and octisalate.

15. The composition of claim 1, wherein the carrier is a porous carrier and at least a portion of the active agent is carried in the pores of the carrier.

16. The composition of claim 1, wherein the carrier is a nonporous carrier and at least a portion of the active agent is adsorbed to a surface of the carrier.

17. The composition of claim 1, wherein the carrier comprises at least one member selected from the group consisting of inorganic porous and nonporous carriers and porous polymeric carriers.

18. The composition of claim 1, wherein the carrier is surface modified with one or more cations selected from the group consisting of aluminum, zinc, magnesium, and nickel cations.

19. The composition of claim 1, wherein the carrier comprises at least one member selected from the group consisting of silica gels and fumed silica.

20. The composition of claim 1, wherein the carrier is a silica gel.

21. A method of preparing the composition according to claim 1, comprising;
melting the hydrophobic barrier-forming material;
mixing the cationic surface active transfer agent and the active agent carried on the carrier into the molten barrier material; and
allowing the combined barrier material, transfer agent and active agent carried on the carrier to solidify.

22. The method of claim 21, further comprising loading the active agent onto the carrier.

23. The method of claim 22, wherein the carrier is a porous carrier and loading the active agent onto the carrier comprises loading at least a portion of the active agent into the pores of the carrier.

24. The method of claim 22, wherein the carrier is a nonporous carrier and loading the active agent onto the carrier comprises adsorbing at least a portion of the active agent to a surface of the carrier.

25. The method of claim 22, further comprising surface treating the carrier with a cation.

26. The method of claim 22, wherein loading the active agent onto the carrier comprises:
mixing a liquid active agent or a soluble, solid active agent with the carrier; and
optionally drying the carrier.

27. A method of preparing the composition according to claim 1, comprising;
melting the hydrophobic barrier-forming material;
mixing the cationic surface active transfer agent into the molten barrier material;
allowing the molten, combined barrier material and transfer agent to solidify;
melting the solid, combined barrier material and transfer agent;
mixing the active agent carried on the carrier into the molten, combined barrier material and transfer agent; and
allowing the combined barrier-forming material, transfer agent and active agent to solidify.

28. A composite, comprising the composition of claim 1 provided on an applicator.

29. A method of preventing, ameliorating and/or treating teeth from discoloration, dental caries, plaque and/or infection, comprising applying an effective amount of the composition of claim 1 to the teeth.

30. A method of preventing, ameliorating and/or treating skin from deleterious effects of sunlight and/or artificial light, comprising applying an effective amount of the composition of claim 1 to the skin.

31. A method of preventing, ameliorating and/or treating skin from insect bites, comprising applying an effective amount of the composition of claim 1 to the skin.
